(19) United States
(12) Patent Application Publication
(10) Pub. No.: US 2005/0267543 A1
(43) Pub. Date: Dec. 1, 2005

(54) Antimicrobial protection for implantable medical device

(75) Inventors: Kenneth T. Heruth, Edina, MN (US); Christopher M. Hobot, Tonka Bay, MN (US); William J. Hooper, Lake Elmo, MN (US); Mark S. Lent, Brooklyn Park, MN (US); Ruchika Singhal, Minneapolis, MN (US); Robert M. Skime, Coon Rapids, MN (US); Randall V. Sparer, Andover, MN (US); Maura G. Donovan, St. Paul, MN (US); Richard D. Ries, Stillwater, MN (US); Kenneth E. Cobian, St. Anthony, MN (US)

Correspondence Address: MEDTRONIC, INC., 710 MEDTRONIC PARKWAY NE, MS-LC340, MINNEAPOLIS, MN, 55432-5604, US

(73) Assignee: MEDTRONIC INC, Minneapolis, MN (US)

(21) Appl. No.: 11/008,664

(22) Filed: Dec. 9, 2004

Related U.S. Application Data
(63) Continuation-in-part of application No. 10/393,121, filed on Mar. 20, 2003.
(60) Provisional application No. 60/529,461, filed on Dec. 12, 2003.
(60) Provisional application No. 60/529,424, filed on Dec. 12, 2003.

(51) Int. Cl. A61N 1/375

(52) U.S. Cl. 607/36

(57) ABSTRACT

An anti-infective covering for an implantable medical device is described. The covering may be a polymeric boot that comprises an anti-infective agent in an amount effective to prevent an infection when implanted in a pocket of a patient. The boot is configured to snuggly engage at least a portion of the implantable medical device. The boot may contain a side hole that allows a housing of the implantable medical device to serve as a return electrode. The boot may be placed about the implantable medical device to render the device anti-infective.

### RELATED APPLICATIONS

This application claims the benefit of priority of and is a Continuation-in-Part application of U.S. application Ser. No. 10/393,121, filed on 20 Mar. 2003 and published as US patent application No. 2004/0186528, which priority application is hereby incorporated herein by reference in its entirety. This application also claims the benefit of priority to U.S. Provisional Patent Application Ser. Nos. 60/529,461 and 60/529,424, both filed on Dec. 12, 2003, which provisional applications are hereby incorporated herein by reference in their entireties.

### FIELD

The present invention relates generally to implantable medical devices (IMDs).

### BACKGROUND

At present, a wide variety of IMDs are commercially released or proposed for clinical implantation that include a housing that is implanted subcutaneously and typically include elongated medical electrical leads or drug delivery catheters that extend from the subcutaneous site to other subcutaneous sites or deeper into the body to organs or other implantation sites. Typically, the IMD includes a battery-powered implantable pulse generator (IPG) that is coupled with electrical medical leads, a battery-powered implantable monitor that may or may not be coupled with electrical medical leads, a battery-powered drug pump coupled with a drug delivery catheter, etc. Such IMDs include implantable cardiac pacemakers, cardioverter/defibrillators having pacing capabilities, other electrical stimulators including spinal cord, deep brain, nerve, and muscle stimulators, drug delivery systems, cardiac and other physiologic monitors, cochlear implants, etc. Typically, the battery-powered component of the IMD is implanted subcutaneously at a surgically prepared site, referred to as a “pocket”. The surgical preparation and initial or replacement IMD implantations are conducted in a sterile field, and the IMD components are packaged in sterile containers or sterilized prior to introduction into the sterile field. However, despite these precautions, there always is a risk of introduction of microbes into the pocket. Surgeons therefore typically apply disinfectant or antiseptic agents to the skin at the surgical site prior to surgery (e.g., Chlorhexidine, Gluconate, Povidone-Iodine, Isopropyl Alcohol, Ethyl Alcohol), directly to the site before the incision is closed (e.g., gentamicin, vancomycin), and prescribe oral antibiotics for the patient to ingest during recovery (e.g., sefuroxin, gentamicin, rifamycin, vancomycin).

Despite these precautions, infections do occur. In addition, once the pocket becomes infected, the infection can migrate along the lead or catheter to the, heart, brain, spinal canal or other location in which the lead or catheter is implanted. Such a migrating infection can become intractable and life-threatening, requiring removal of the IMD in the pocket and associated devices, such as leads and catheters. Removal of a chronically implanted lead or catheter can be difficult and dangerous. Aggressive systemic drug treatment is also provided to treat the infection. To prevent pocket infection and thus the ability of infection migration along a lead or catheter, there is a need to impart antimicrobial activity to the IMD residing in the pocket itself.

There is long history of the actual or proposed use of antimicrobial agents coated on IMDs for prevention of infection. However, applying coatings to surfaces of IMDs intended for long-term implantation can be problematic because the coatings can degrade and slough away over time. This may be particularly problematic with IMDs configured to be implanted in the pocket, which IMDs may contain metallic surfaces. Such IMDs, e.g., such as neurostimulatory pulse generators, cardiac pacemakers, drug infusion pumps, and the like, containing metallic surfaces can be more difficult to coat than polymeric surfaces. As such, there is a need to impart antimicrobial activity to active IMDs residing in subcutaneous pockets, where the vehicle containing the antimicrobial activity can withstand long-term implantation.

### SUMMARY

Various embodiments of the invention are directed to providing a simple, effective and long lasting anti-microbial agent into the subcutaneous implantation pocket that is surgically prepared to receive an IMD. This may be accomplished by disposing about the IMD a covering comprising an anti-infective agent. The covering may be a boot, jacket, etc. The anti-infective agent is present on the surface of the covering or is eluted from the covering in an amount sufficient to prevent infection in a subcutaneous pocket into which the IMD is implanted. The covering may be conformed to the shape of the IMD implanted into the pocket and may be attached to or detached from the IMD. In an embodiment, the covering is a polymeric boot that fits around at least a portion of an outer housing of the IMD.

Polymeric boots have been proven over long-term clinical use to not degrade significantly in the body despite the fact that they are relatively thin. Therefore, it is expected that anti-infective agent dispersed through the thin wall of the anti-microbial pad or boot component or other component will be beneficially present or released over time.

By using coverings as described herein, as opposed to coatings, it is not necessary for manufacturers to commit to manufacturing and clinical buyers to stock redundant models of expensive IMDs, one model with the anti-infective polymeric component and one without the anti-microbial polymeric component. Once it is determined that an IMD having anti-infective properties is desired, the coating may be placed about the IMD by the manufacturer, the consumer, or the user.

This summary of the invention has been presented here simply to point out some advantages over the prior art and is not intended to operate in any manner as a limitation on the interpretation of claims that are presented initially in the patent application and that are ultimately granted.

These and other advantages will be more readily understood from the following detailed description, when considered in conjunction with the drawings.

### BRIEF DESCRIPTION OF THE DRAWINGS

FIG. 1 is a schematic view of an implantable medical device implanted subcutaneously in a patient's thoracic region, having a polymeric boot comprising an anti-infective agent fitted over the device.

FIG. 2 is a plan view of the polymeric boot of FIG. 1.

FIG. 3 is a side-cross-section view of the boot taken along lines 3-3 of FIG. 2.

FIG. 4 is a top view of the boot of FIG. 2.

FIG. 5 is a schematic view of an implantable medical device implanted subcutaneously in a patient's thoracic region, having a polymeric boot comprising an anti-infective agent fitted over the device and having a further boot fitted over or attached to the non-conducting side of the device.

FIG. 6 is a schematic view of an implantable medical device including two modules implanted subcutaneously across the patient's thorax and tethered together, each module having a boot comprising an anti-infective agent fitted over the device.

FIG. 7 is a schematic view of an implantable medical device implanted subcutaneously in a patient's abdominal region having a boot comprising an anti-infective agent fitted over the device.

FIG. 8 is a schematic view of an implantable medical device implanted subcutaneously in a patient's abdominal region having a boot comprising an anti-infective agent fitted over the device.

FIG. 9 is a schematic view of an implantable medical device implanted subcutaneously in a patient's pectoral region having a boot comprising an anti-infective agent fitted over the device.

FIG. 10 is a schematic view of an implantable medical device implanted subcutaneously in a patient's pectoral region having a boot comprising an anti-infective agent fitted over the device.

FIG. 11 is a schematic view of an implantable medical device implanted subcutaneously in a patient's pectoral region having a boot comprising an anti-infective agent fitted over the device.

FIG. 12 is a schematic partial view of an exemplary implantable medical device depicting a connector header in partial cross-section and an exemplary lead connector assembly adapted to be fitted into a connector bore, wherein selected ones or all of polymeric components of the connector header and/or the lead connector assembly comprise an anti-infective agent.

FIG. 13 is a perspective view of a subcutaneously implantable electrode wherein selected ones or all of the polymeric components of the electrode comprise an anti-infective agent.

The drawings are not necessarily to scale.

### DETAILED DESCRIPTION

In the following detailed description, references are made to illustrative embodiments of methods and apparatus for carrying out the invention. It is understood that other embodiments can be utilized without departing from the scope of the invention.

Anti-Infective Agents

Any anti-infective agent may be incorporated in or on a covering configured to be disposed about an IMD. Preferably, the anti-infective agent is present in or on the covering, or may be eluted from the covering, in an amount sufficient to prevent an infection from forming in a pocket into which the IMD is implanted. It is also desirable that the anti-infective agent, in the concentration present in the covering, be nontoxic when implanted in the pocket. It will be understood that more than one anti-infective agent may be present in or on the covering. As used herein, “anti-infective agent” means an agent that prevents an infection. Anti-infective agents include agents that kill or inhibit the growth of a microbe or a population of microbes. Non-limiting examples of such agents include antibiotics and antiseptics.

Any antibiotic suitable for use in a human may be used in accordance with various embodiments of the invention. As used herein, “antibiotic” means an antibacterial agent. The antibacterial agent may have bateriostatic and/or bacteriocidal activities. Nonlimiting examples of classes of antibiotics that may be used include tetracyclines (e.g. minocycline), rifamycins (e.g. rifampin), macrolides (e.g. erythromycin), penicillins (e.g. nafcillin), cephalosporins (e.g. cefazolin), other beta-lactam antibiotics (e.g. imipenem, aztreonam), aminoglycosides (e.g. gentamicin), chloramphenicol, sufonamides (e.g. sulfamethoxazole), glycopeptides (e.g. vancomycin), quinolones (e.g. ciprofloxacin), fusidic acid, trimethoprim, metronidazole, clindamycin, mupirocin, polyenes (e.g. amphotericin B), azoles (e.g. fluconazole) and beta-lactam inhibitors (e.g. sulbactam). Nonlimiting examples of specific antibiotics that may be used include minocycline, rifampin, erythromycin, nafcillin, cefazolin, imipenem, aztreonam, gentamicin, sulfamethoxazole, vancomycin, ciprofloxacin, trimethoprim, metronidazole, clindamycin, teicoplanin, mupirocin, azithromycin, clarithromycin, ofloxacin, lomefloxacin, norfloxacin, nalidixic acid, sparfloxacin, pefloxacin, amifloxacin, enoxacin, fleroxacin, temafloxacin, tosufloxacin, clinafloxacin, sulbactam, clavulanic acid, amphotericin B, fluconazole, itraconazole, ketoconazole, and nystatin. Other examples of antibiotics, such as those listed in Sakamoto et al., U.S. Pat. No. 4,642,104, which is herein incorporated by reference in its entirety, may also be used. One of ordinary skill in the art will recognize other antibiotics that may be used.

It is desirable that the antibiotic(s) selected kill or inhibit the growth of one or more bacteria that are associated with infection following surgical implantation of a medical device. Such bacteria are recognized by those of ordinary skill in the art and include Stapholcoccus aureus and Staphlococcus epidermis. Preferably, the antibiotic(s) selected are effective against strains of bacteria that are resistant to one or more antibiotic.

To enhance the likelihood that bacteria will be killed or inhibited, it may be desirable to combine one or more antibiotic. It may also be desirable to combine one or more antibiotic with one or more antiseptic. It will be recognized by one of ordinary skill in the art that antimicrobial agents having different mechanisms of action and/or different spectrums of action may be most effective in achieving such an effect. In a particular embodiment, a combination of rifampin and minocycline is used.

Any antiseptic sutable for use in a human may be used in accordance with various embodiments of the invention. As used herein, “antiseptic” means an agent capable of killing or inhibiting the growth of one or more of bacteria, fungi, or viruses. Antiseptic includes disinfectants. Nonlimiting examples of antiseptics include hexachlorophene, cationic bisiguanides (i.e. chlorhexidine, cyclohexidine) iodine and iodophores (i.e. povidone-iodine), para-chloro-meta-xylenol, triclosan, furan medical preparations (i.e. nitrofurantoin, nitrofurazone), methenamine, aldehydes (glutaraldehyde, formaldehyde), silver sulfadiazine and alcohols. One of ordinary skill in the art will recognize other antiseptics.

It is desirable that the antiseptic(s) selected kill or inhibit the growth of one or more microbe that are associated with infection following surgical implantation of a medical device. Such bacteria are recognized by those of ordinary skill in the art and include Stapholcoccus aureus, Staphlococcus epidermis, Pseudomonus auruginosa, and Candidia.

To enhance the likelihood that microbes will be killed or inhibited, it may be desirable to combine one or more antiseptics. It may also be desirable to combine one or more antiseptics with one or more antibiotics. It will be recognized by one of ordinary skill in the art that antimicrobial agents having different mechanisms of action and/or different spectrums of action may be most effective in achieving such an effect. In a particular embodiment, a combination of chlorohexidine and silver sulfadiazine is used.

An anti-infective agent, such as an antibiotic or antiseptic, may be present in the covering at any concentration effective, either alone or in combination with another anti-infective agent, to prevent an infection within a pocket into which the covering is implanted. Generally, an antiseptic agent may be present in the covering at a range of between about 0.5% and about 20% by weight. For example, the anti-infective agent may be present in the covering at a range of between about 0.5% and about 15% by weight or between about 0.5% and about 10% by weight.

Covering

An embodiment of the invention provides a covering configured to be placed about at least a portion of an implantable medical device. The covering may be in the form of a boot, jacket, gauze, wrap and the like. The covering is formed of a polymeric material into or onto which an anti-infective agent is incorporated. Any polymeric material may be used. Preferably the polymeric material is biocompatible and is capable of presenting or eluting the anti-infective agent to the implant pocket in an amount effective to prevent an infection.

Examples of suitable polymeric materials that may be used to form the covering include organic polymers such as silicones, polyamines, polystyrene, polyurethane, acrylates, polysilanes, polysulfone, methoxysilanes, and the like. Other polymers that may be utilized include polyolefins, polyisobutylene and ethylene-alphaolefin copolymers; acrylic polymers and copolymers, ethylene-covinylacetate, polybutylmethacrylate; vinyl halide polymers and copolymers, such as polyvinyl chloride; polyvinyl ethers, such as polyvinyl methyl ether; polyvinylidene halides, such as polyvinylidene fluoride and polyvinylidene chloride; polyacrylonitrile, polyvinyl ketones; polyvinyl aromatics, such as polystyrene, polyvinyl esters, such as polyvinyl acetate; copolymers of vinyl monomers with each other and olefins, such as ethylene-methyl methacrylate copolymers, acrylonitrile-styrene copolymers, ABS resins, and ethylene-vinyl acetate copolymers; polyamides, such as Nylon 66 and polycaprolactam; polycarbonates; polyoxymethylenes; polyimides; polyethers; epoxy resins; polyurethanes; rayon; rayon-triacetate; cellulose; cellulose acetate, cellulose butyrate; cellulose acetate butyrate; cellophane; cellulose nitrate; cellulose propionate; cellulose ethers; carboxymethyl cellulose; polyphenyleneoxide; and polytetrafluoroethylene (PTFE). In an embodiment the covering comprises silicone. In an embodiment, the covering comprises polyurethane.

An anti-infective agent may be incorporated into or on the polymeric covering using any known or developed technique. For example, the anti-infective agent may be adhered to a surface of the covering, adsorbed into the covering, or compounded into the polymeric material that forms the covering. Accordingly, the anti-infective material may be embedded, coated, mixed or dispersed on or in the material of the covering. In various embodiments, the anti-infective agent may be incorporated into the polymeric covering as taught by U.S. Pat. Nos. 5,217,493 or 5,624,704.

In an embodiment, the covering is a boot. The boot may be molded into a shape to conform to that of at least a portion of an IMD using known or developed techniques. The IMD may be an active IMD, such as a cardiac pacemaker, a cardioverter/defibrillators, a neurostimulator, a drug infusion pump, and the like.

The remainder of this description may refer specifically to a silicone rubber boot 15, 215, 335, 340, etc. into which an anti-microbial metal ion zeolite is compounded. However, it will be understood that any covering may be substituted for the boot 15 and that any anti-infective agent may be substituted for the metal ion zeolite.

In an embodiment the covering is any covering as described herein, with the proviso that the anti-infective agent is not a metal ion zeolite.

In an embodiment the covering is any covering as describe herein, with the proviso that if the anti-infective agent is a metal ion zeolite, then the metal zeolite is not compounded into the covering.

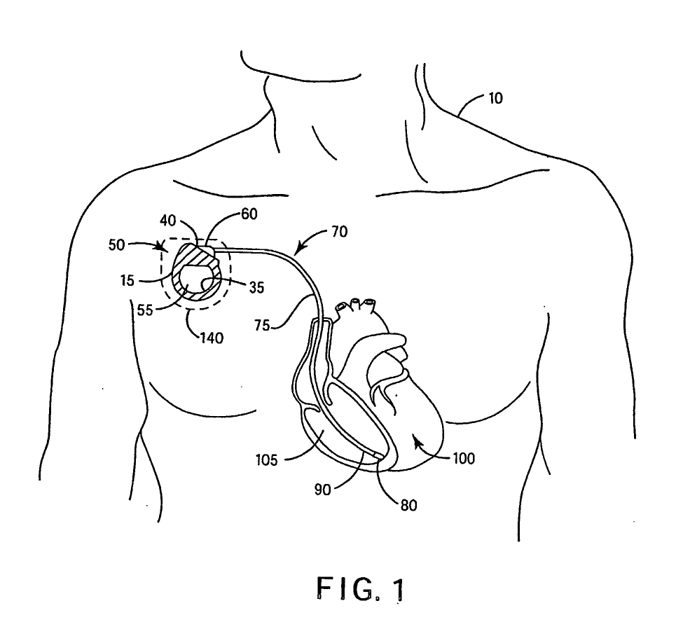

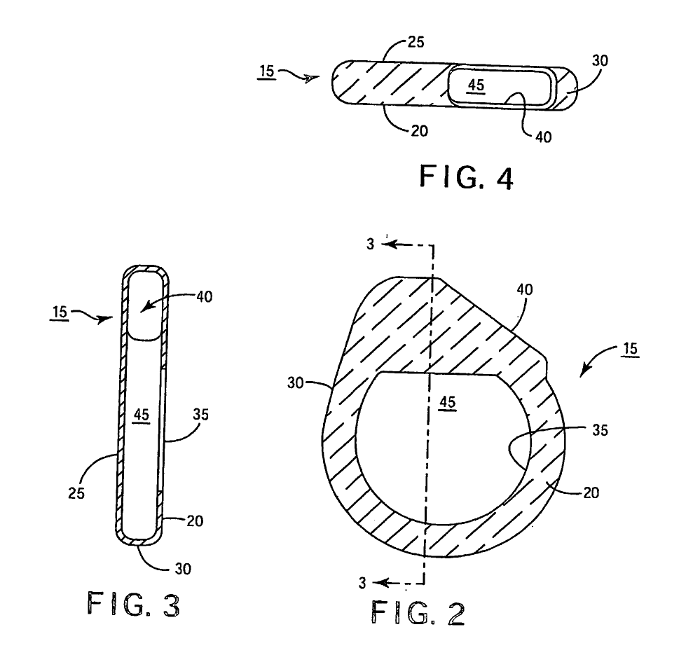

In an embodiment of a detachable, elastic, boot 15 that is compounded of silicone rubber and the preferred anti-microbial metal ion zeolite and molded in a shape to be tatted over an IPG or monitor 50 implanted in patient 10 is depicted in FIGS. 1-4. The boot 15 has first and second major boot sides 20 and 25 joined by a mutual boot edge 30 defining a boot cavity 45. A side opening 35 through major boot side 20 and an edge opening 40 through a segment of boot edge 30 are provided.

The boot 15 is fitted over the housing 55 and connector block 60 of the exemplary IPG or monitor and inserted into a subcutaneous pocket 140 at a distance from the heart 100 as shown in FIG. 1. The fitted boot 15 provides the anti-microbial protection in the subcutaneous implantation pocket 140 while leaving at least a portion of the housing 55 of IPG/monitor 50 exposed through side opening 35. Preferably, the size and shape of the side opening fits within a circle having a diameter of the zone of inhibition of the one or more anti-infective agents in or on the boot 15. In an embodiment, the diameter of the zone of inhibition is determined at 30 days post-implantation. In an embodiment, the diameter of the zone of inhibition is determined at 90 days post-implantation. If such a sized and shaped side opening 35 is too small for its intended purposes, more than one side opening 35, each having a size and shape fitting within a circle having a diameter of the zone of inhibition of the one or more anti-infective agents in or on the boot 15 may be employed.

The IPG 50 depicted in FIG. 1 as a ventricular pacemaker IPG or hemodynamic monitor that is coupled to a cardiac lead 70 extending from a connection with connector block 60 into the heart 100 through a conventional transvenous route. The cardiac lead comprises an active or cathodal pace/sense electrode 80 at the distal end of lead body and optionally comprises a pressure transducer 90 proximal to pace/sense electrode both disposed in this instance in the right ventricle 105 of heart 100. The housing of IPG 50 is hermetically sealed and formed of a conductive metal that is electrically connected to pacing and/or sensing circuitry within housing 55 to function as an indifferent or anodal pace/sense electrode 85 that is exposed by side opening 35.

The housing 55 and connector block 60 of IPG/monitor 50 can take any shape known in the art, and that shape dictates the shape and dimensions of the boot 15. The specifications and operating modes and other characteristics of the pacemaker IPG and the cardiac lead(s) coupled therewith can correspond to any of those known in the art. The monitor can correspond to the Medtronic® CHRONICLE® IHM (implantable hemodynamic monitor) that is coupled through a cardiac lead of the type described in commonly assigned U.S. Pat. No. 5,564,434 having capacitive blood pressure and temperature sensors as well as at least one EGM sense electrode.

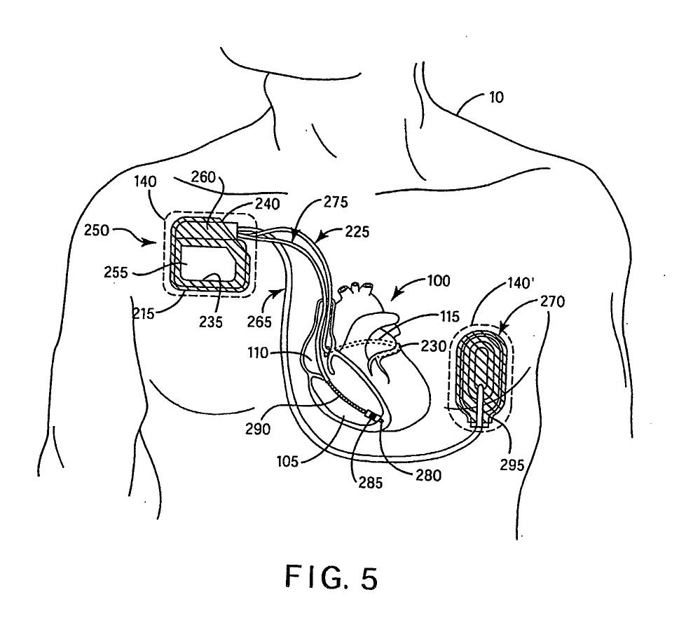

The IPG/monitor 50 is slipped through the side opening 35 and the connector block 60 is oriented to be exposed through the edge opening 40. It will also be understood that the side opening 35 is necessary to expose the housing 55 for use as a remote indifferent stimulating and/or sensing electrode in either of a unipolar pacemaker IPG/monitor 50 or in a bipolar pacemaker IPG/monitor also having the capability of monitoring the far field EGM. The boot 15 having such a side opening 35 can still be efficaciously used over a typical bipolar pacemaker IPG/monitor not having such a far held sensing capability. These features of the boot 15 are applicable to the remaining boot embodiments illustrated in FIGS. 5-10.

An embodiment of a detachable, elastic, boot 215 that is compounded of silicone rubber and the preferred anti-microbial metal ion zeolite and molded in a shape to be fitted over a rectilinear ICD IPG 250 implanted in patient 10 is depicted in FIG. 5. The boot 215 is also formed of first and second major boot sides joined by a mutual boot edge defining a side opening 235 through major boot side and an edge opening 240 through a segment of the boot edge.

The boot 215 is fitted over the housing 255 and connector block 260 of the exemplary ICD IPG 250 and inserted into a subcutaneous pocket 140 at a distance from the heart 100 as shown in FIG. 5. The fitted boot 215 provides the anti-microbial protection in the subcutaneous implantation pocket 140 while leaving at least a portion of the housing 255 of ICD IPG 250 exposed through side opening 235. The exposed portion of the housing 255 may be employed as one electrode.

The ICD IPG 250 depicted in FIG. 5 is coupled to an exemplary set of leads extending to pace/sense electrodes and electrodes. It will be understood that not all of the depicted leads and that other combinations of leads can be connected to the ICD IPG 250. In this particular instance, a right ventricular (RV) lead 275 extends from a connection with connector block 260 into the right ventricle 105 of the heart 100 through a conventional transvenous route. The RV lead 275 comprises active or cathodal pace/sense electrode and fixation helix 280 at the distal end of the lead body, a more proximally located, ring-shaped, indifferent or anodal pace/sense electrode 285, and an elongated electrode 290. A coronary sinus (CS) lead 225 extends from a connection with connector block 260 to an elongated electrode 230 disposed in the coronary sinus or great vein 115 of the heart 100 through a conventional transvenous route.

A further lead 265 extends subcutaneously from a connection with connector block 260 to a rectilinear, pad-shaped, electrode 270 disposed in a further subcutaneous pocket 140′ selected by the surgeon to optimally apply shock therapies between selected pairs of the electrodes 230, 255, 270, and 290.

Typically the rectilinear electrode 270 is formed of a flexible silicone rubber or polyurethane pad supporting a electrode surface or array on one major side disposed toward heart 100 and a non-conductive side disposed toward the skin. A further detachable, elastic, boot 295 that is compounded of silicone rubber and the preferred anti-microbial metal ion neolith and molded in a shape to be fitted over the non-conductive major side of the rectilinear electrode 770 is shown in FIG. 5.

The boot 295 can be affixed by sutures or other means to the silicone rubber or polyurethane pad to ensure that it does not move or detach from the non-conductive side within the pocket 140′.

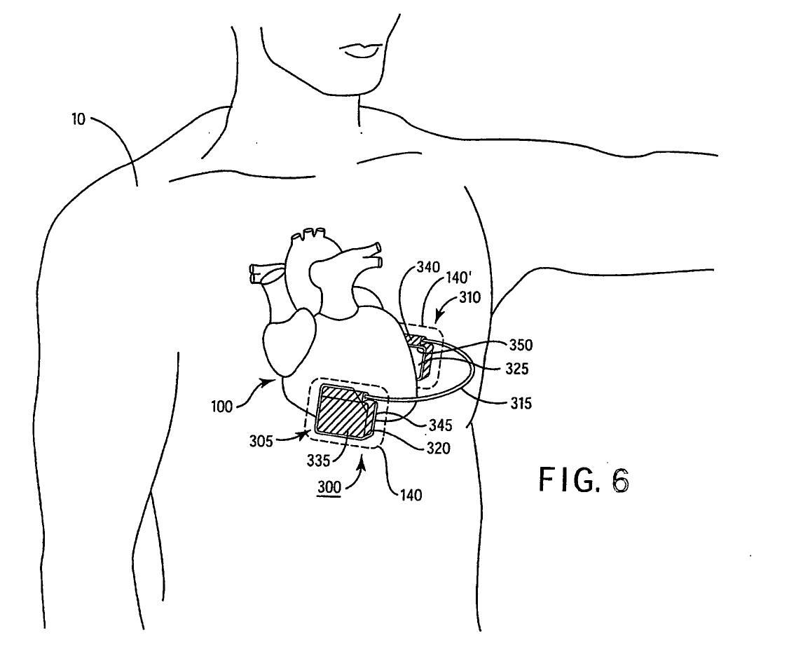

More recently, it has been proposed that all components of an ICD be implanted subcutaneously distributed between two or more electrode bearing; modules implanted in subcutaneous pockets 140, 140′ around the thorax to deliver shock therapies between them and through the heart. Such ICDs are disclosed in U.S. Pat. Nos. 5,255,692, 5,314,451, and 5,342,407 and in U.S. patent application Publication Nos. 2002/0042634 and 2002/0035377. Such an arrangement is depicted in FIG. 6 wherein the ICD 300 comprises first and second schematically depicted, hermetically sealed ICD IPG modules 305 and 310 tethered together by a cable 315.

First and second electrodes 320 and 325 are supported on one side of the ICD IPG modules 305 and 310, respectively, that are intended to be implanted in the subcutaneous pockets 140, 140′ facing the heart 100 and one another.

The hermetically sealed ICD IPG module 305 encloses the electronic sensing, pacing, and circuitry, including the relatively bulky high voltage capacitors that are charged and discharged to deliver shocks, as well as a low voltage battery employed for powering the circuitry and the delivered pacing pulses. The second hermetically sealed ICD IPG module 310 encloses a relatively bulky high power battery as well as a switch to enable selective connection with the high voltage capacitor charging circuitry within the first ICD IPG module 305 in the manner described in the above referenced '451 patent. The cable 315 encases conductors distributing power from the battery and exchanging signals and commands between circuitry in the first and second ICD IPG modules 305 and 310.

First and second detachable, elastic, boots 335 and 340 that are each compounded of silicone rubber and the preferred anti-microbial metal ion zeolite and molded in a shape to be fitted over the respective first and second ICD IPG modules 305 and 310 implanted in patient 10 are also depicted in FIG. 6. The boots 335 and 340 have openings 345 and 350 in the major sides thereof that expose the first and second respective electrodes 320 and 325.

The first and second hermetically sealed ICD IPG modules 305 and 310 bearing the first and second detachable, elastic, boots 335 and 340 are preferably implanted subcutaneously in posterior and anterior positions through a single skin incision intermediate the illustrated posterior and anterior positions. Tunneling tools would be employed to displace the tissue and advance the first and second hermetically sealed housings to the depicted sites or other selected sites around the thorax. Tissue adhesive may be employed to secure the first and second hermetically sealed ICD IPG modules 305 and 310 bearing the first and second detachable, elastic, boots 335 and 340 at the sites and prevent migration. Alternatively, the sites may be exposed through minimal surgical exposures, and the first and second hermetically sealed ICD IPG modules 305 and 310 bearing the first and second detachable, elastic, boots 335 and 340 can be sutured at the sites through the boots 335 and 340 to prevent migration.

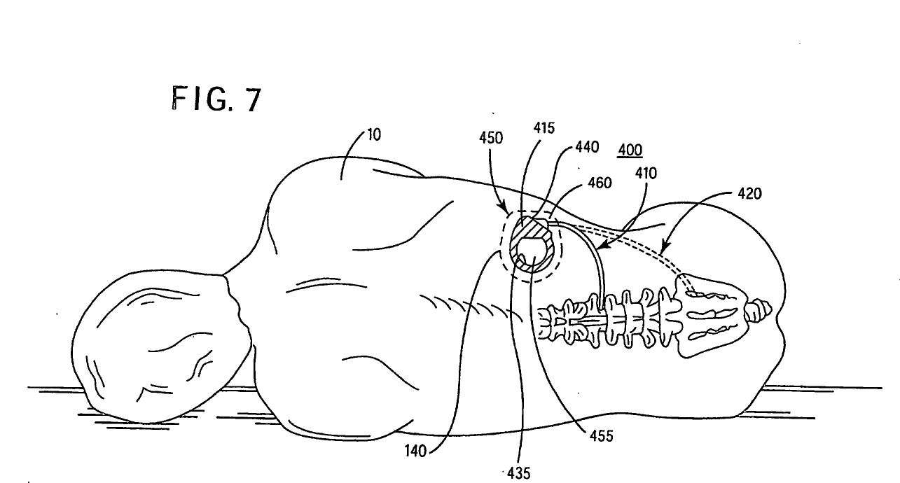

Therapeutic administration of pain suppressing electrical stimulation into the intraspinal space, that is to either the epidural space or to the intrathecal space, is also known in the art as illustrated in FIG. 7. Three meningeal sheaths that are continuous with those which encapsulate the brain within the enclosure by the vertebral canal for the spinal cord by the bones of the vertebrae surround the spinal cord. The outermost of these three meningeal sheaths is the dura matter, a dense, fibrous membrane which anteriorally is separated from the periosteum of the vertebral by the epidural space. Posterior to the dura matter is the subdural space. The subdural space surrounds the second of the three meningeal sheaths, the arachnoid membrane, which surround the spinal cord. The arachnoid membrane is separated from the third meningeal sheath, the pia mater, by the subarachnoid or intrathecal space. The subarachnoid space is filled with CSF. Underlying the pia mater is the spinal cord. Thus the progression proceeding inwards or in posterior manner from the vertebra is the epidural space, dura mater, subdural space, arachnoid membrane, intrathecal space, pia matter and spinal cord.

An exemplary spinal cord stimulation (SCS) system 400 comprising a neurostimulator SCS IPG 450, an SCS lead 410, and a detachable, elastic, boot 415 that is each compounded of silicone rubber and the preferred anti-microbial metal ion zeolite and molded in a shape to be fitted over the housing and connector of the neurostimulator IPG 450 is depicted implanted in patient 10 in FIG. 7. The neurostimulator IPG 450 may comprise the Medtronic® Itrel® 3, Synergy™ or Synergy Versitrel™ neurostimulator, and the SCS lead 410 may comprise the Medtronic® Pisces Z Quad lead.

Therapeutic administration of stimulation of the sacral nerves to control bladder function or treat sexual dysfunction is also alternatively illustrated in FIG. 7 by the sacral nerve stimulation lead 420 depicted in dotted lines extending from the neurostimulator IPG 450 and detachable, elastic, boot 415 into a foramen of the sacrum. In this case, the neurostimulator IPG 450 may comprise the Medtronic® InterStim® Neurostimulator Model 3023. In one embodiment, a sacral nerve stimulation lead 420 bearing one or a plurality of distal stimulation electrodes are percutaneously implanted through the dorsum and the sacral foremen of the sacral segment S3 for purposes of selectively stimulating the S3 sacral nerve. The distal electrode(s) is positioned using a hollow spinal needle through a foremen (a singular foramina) in the sacrum. The electrode is secured by suturing the lead body in place, and the lead body is tunneled subcutaneously to the implant site of the neurostimulator IPG 450 within the boot 415.

The detachable, elastic, boot 415 corresponds to the detachable, elastic, boot described above with respect to FIGS. 1-4. It will be understood that the actual shape of such commercially available neurostimulator IPGs may differ from the exemplary shape of neurostimulator IPG 450 shown in FIG. 7, and that boot 415 is molded to conform to the actual shape. Again, the boot 415 has a major side opening 435 exposing the housing 455 of the IPG 450 that can function as an indifferent stimulation electrode in conjunction with a stimulation electrode or electrodes along the distal end segment of the SCS lead 410 disposed within the intraspinal space and obscured from view. The boot 415 also has an edge opening 440 enabling access to the connector block 460.

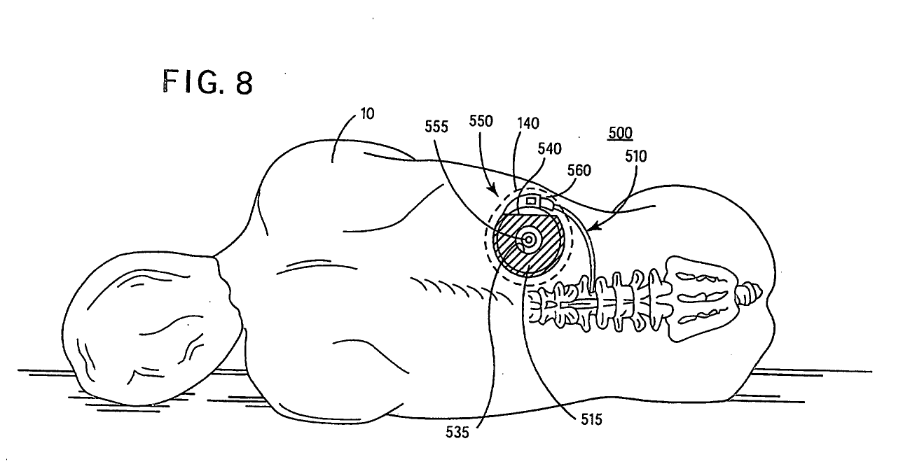

Therapeutic administration of pain suppression or therapeutic drugs into the intraspinal space as also known in the prior art is illustrated in FIG. 8. Administration of a drug directly to the intrathecal space can be by either spinal tap injection or by catheterization.

Intrathecal drug administration can avoid the inactivation of some drugs when taken orally as well and the systemic effects of oral or intravenous administration. Additionally, intrathecal administration permits use of an effective dose that is only a fraction of the effective dose required by oral or parenteral administration. Furthermore the intrathecal space is generally wide enough to accommodate a small catheter, thereby enabling chronic drug delivery systems. Thus, it is known to treat spasticity by intrathecal administration of baclofen. Additionally, it is known to combine intrathecal administration of baclofen with intramuscular injections of botulinum toxin for the adjunct effect of intramuscular botulinum for reduced muscle spasticity. Furthermore, it is known to treat pain by intraspinal administration of the opioids morphine and fentanyl. A drug pump is required because the antinociceptive or antispasmodic drugs in current use have a short duration of activity and must therefore be frequently re-administered, which re-administration is not practically carried out by daily spinal tap injections. The drug pump is surgically placed under the skin of the patient's abdomen. One end of a catheter is connected to the pump, and the other end of the catheter is threaded into a CSF filled subarachnoid or intrathecal space in the patient's spinal cord. The implanted drug pump can be programmed for continuous or intermittent infusion of the drug through the intrathecally located catheter.

Thus a fully implantable intrathecal drug delivery system 500, e.g., the Medtronic® SynchroMed® EL Infusion System, comprising a programmable SynchroMed® drug pump 550 and a drug delivery catheter 510, is depicted in FIG. 8.

A detachable, elastic, boot 515 that is compounded of silicone rubber and the preferred anti-microbial metal ion zeolite and molded in a shape to be fitted over the housing and connector of the drug pump 550 is depicted implanted in patient 10 in FIG. 7. Again, the boot 515 has a major side opening 535 in this case exposing a drug fill port 555 for percutaneously refilling a drug chamber within the drug pump 550 in a manner well known in the art. The boot 515 also has an edge opening 540 enabling access to the connector block 560 that the drug delivery catheter 510 is attached to.

The drug pump 550 and boot 515 encasing the drug pump 550 are implanted just under the skin of the abdomen in a prepared subcutaneous pocket 140 so that the drug fill port is oriented outward to enable access to the drug fill port 555.

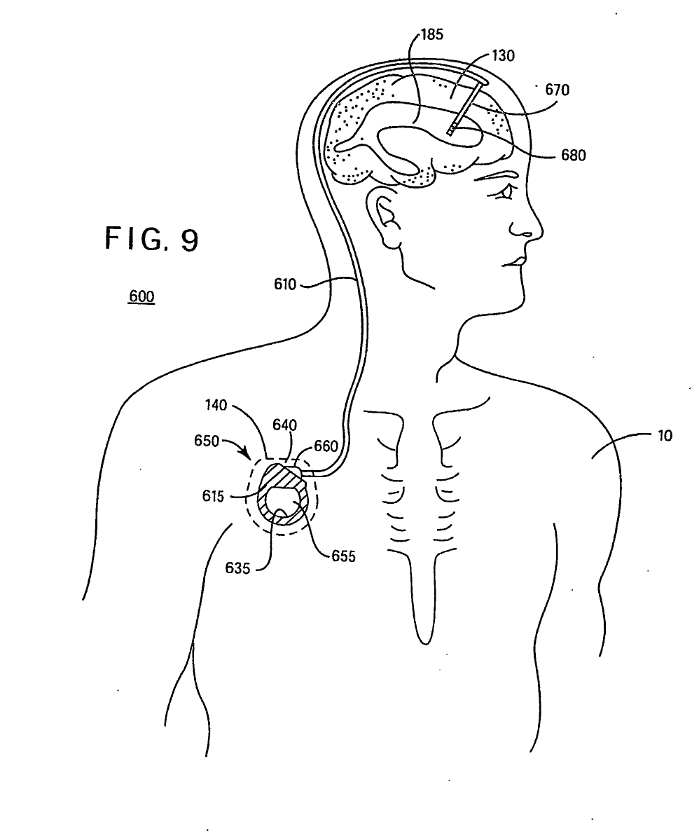

Turning to FIG. 9, it schematically illustrates the delivery of Medtronic® Activa® Tremor Control Therapy or Parkinson's Control Therapy to a patient 10 for controlling essential tremors and those associated with Parkinson's disease. The Activate Therapy is delivered by an deep brain stimulator similar to a cardiac pacemaker, that uses mild electrical stimulation delivered by electrodes implanted in the brain to block the brain signals that cause tremor.

The Activa® Tremor Control System stimulates targeted cells in the thalamus the brain's message relay center—via electrodes that are surgically implanted in the brain and connected to a neurostimulator IPG implanted near the collarbone. In the treatment of Parkinson's tremors, the electrodes are located at the subthalamic nucleus (STN) or globus pallidus interna (GPI) that control movement and muscle function. A lead with tiny electrodes is surgically implanted at these sites in the brain and connected by an extension that lies under the skin to a neurostimulator IPG implanted near the collarbone. The electrical stimulation can be non-invasively adjusted to meet each patient's needs.

The implanted components of the Activa® System 600 depicted in FIG.9 include the Medtronic® Itrel® II Model 7424 neurostimulator IPG 650, a DBS™ lead 670 and an extension 610 that connects the lead 670 to the neurostimulator IPG 650.

The lead 670 is implanted using a stereotactic headframe designed to keep the head stationary and help guide the surgeon in the placement of the lead 670 into the brain 130 to dispose the electrodes 680 at the desired site 135. The brain 130 and the placement of the lead 670 is imaged using CT (computed tomography) or MRI (magnetic resonance imaging) equipment. The Model 3387 DBS™ lead, with a plurality of widely spaced electrodes, and the Model 3389 DBS™ lead, with a plurality of narrowly spaced electrodes, provide physician options for precise placement and stimulation selectivity. Other components of the Activate System 60 include a neurostimulator control magnet, neurological test stimulator, physician programmer, lead frame kits, and Memory Mod software cartridge.

A detachable, elastic, boot 615 that is compounded of silicone rubber and the preferred anti-microbial metal ion zeolite and molded in a shape to be fitted over the housing and connector block of the neurostimulator IPG 650 is depicted implanted in patient 10 in FIG. 9. Again, the boot 615 has a major side opening 635 and an edge opening 640 enabling access to the connector block 660 that the lead extension 610 is attached to. The neurostimulator IPG 650 and boot 615 encasing the neurostimulator IPG 650d are implanted just under the skin of the upper thorax in a prepared subcutaneous pocket 140. The exposed surface of the bipolar neurostimulator housing 655 can be employed as a stimulation electrode in this instance.

An implantable infusion pump (IIP) comprising an implantable drug pump and: catheter is disclosed in commonly assigned U.S. Pat. Nos. 5,643,207 and 5,782,798 for dispensing pancreatic polypeptide blockers and other drugs that decrease sensations of hunger and increase satiety into particular sites in the brain through a distal catheter segment that is implanted through the skull and extends to the specific sites. The delivery of other appetite influencing drugs directly into the brain for increasing appetite to treat anorexia is also proposed in the '207 patent. The drug that is dispensed from the infusion pump coupled to the catheter through the catheter lumen and into the brain is expected to induce or increase the feeling of satiety to treat: obesity by reducing caloric intake or to increase feelings of hunger to treat anorexia by increasing caloric intake. The system of the '798 patent can also be employed to apply electrical stimulation to the brain through catheter borne electrodes and conductors to increase feelings of satiety to treat obesity or to decrease feelings of satiety to treat anorexia presumably either with or without delivery of the identified drugs.

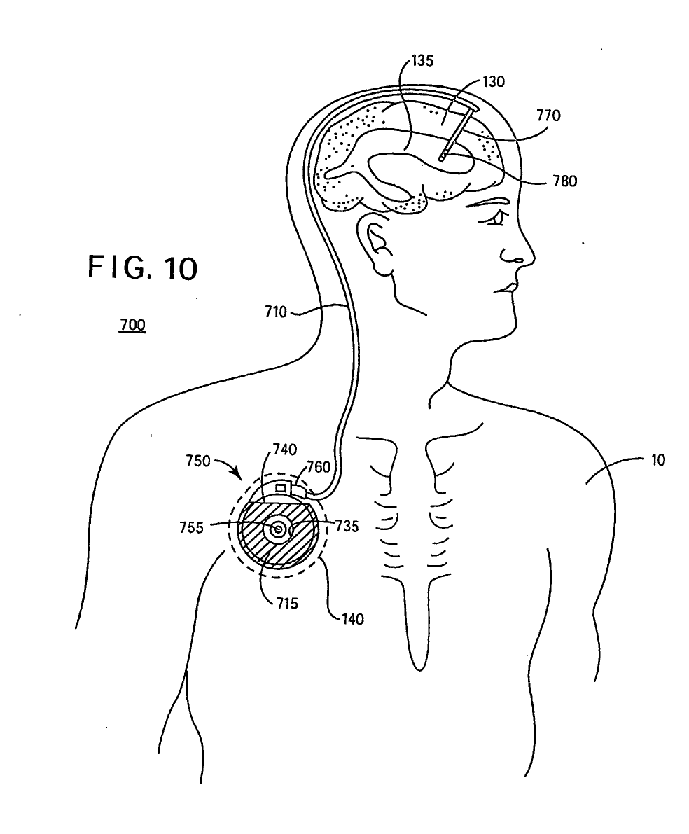

Such an implantable deep brain drug delivery system 700 is depicted in FIG. 10 comprising an implantable drug pump 750 and catheter 710 for dispensing pancreatic polypeptide blockers and other drugs that decrease sensations of hunger and increase satiety through catheter ports 780 into a particular site 135 in the brain through a distal catheter segment 770 that is implanted through the skull and extends to the specific site 135. The implantable drug pump 750 can comprise a programmable SynchroMed® drug pump 750. A detachable, elastic, boot 715 that is compounded of silicone rubber and the preferred anti-microbial metal ion zeolite and molded in a shape to be fitted over the housing and connector of the drug pump 750 is depicted implanted in patient 10 in FIG. 10. Again, the boot 715 has a major side opening 735 in this case exposing a drug fill port 755 for percutaneously refilling a drug chamber within the drug pump 750 in a manner well known in the art. The boot 715 also has an edge opening 740 enabling access to the connector block 760 that the drug delivery catheter 710 is attached to. The drug pump 750 and boot 715 encasing the drug pump 750 are implanted just under the skin of the thorax in a prepared subcutaneous pocket 140 so that the drug fill port is oriented outward to enable access to the drug fill port 755.

An implantable EGM monitor for recording the cardiac electrogram from electrodes remote from the heart is disclosed in commonly assigned U.S. Pat. No. 5,331,966 and PCT publication WO 98/02209 and is embodied in the Medtronic® REVEAL® Model 9526 Insertable Loop Recorder having spaced housing EGM electrodes employed with a Model 6191 patient activator and a Model 9790 programmer. Such implantable monitors when implanted in patients suffering from cardiac arrhythmias or heart failure accumulate date and time stamped data that can be of use in determining the condition of the heart over an extended period of time and while the patient is engaged in daily activities. A wide variety of other IMDs have been proposed to monitor many other physiologic conditions as set forth in U.S. Pat. No. 6,221,011.

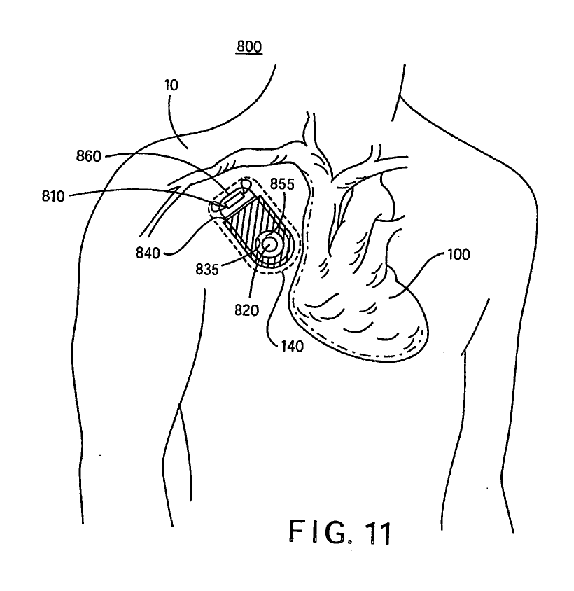

Therefore, a REVEAL® Insertable Loop Recorder 850 is depicted in FIG. 11 implanted in a subcutaneous pocket 140 in the thorax of patient 10. The Insertable Loop Recorder 850 comprises a hermetically sealed housing 855 enclosing the monitoring circuitry, battery, telemetry antenna, and other components and a header 860 that supports a sense electrode 810 coupled to the a sense amplifier via a feedthrough extending through the housing 855 and has a pair of suture holes extending through it. An electrically un-insulated portion of the housing 855 that is coupled with the sense amplifier provides a second sense electrode 820. A detachable, elastic, boot 815 that is compounded of silicone rubber and the preferred anti microbial metal ion zeolite and molded in a shape to be fitted over at least the housing 855. Again, the boot 815 has a major side opening 835 exposing the sense electrode 820 and an edge opening 840 enabling insertion of the housing 855 into the boot 815.

The boot 815 may be shaped to extend over at least the portions of the header 860 having the suture holes to enable using the same sutures to secure the boot to the Insertable Loop Recorder 850 and the Insertable Loop Recorder 850 to subcutaneous tissue.

Thus, a variety of subcutaneously implanted IMDs have been described having a variety of uses and shapes that are implanted in subcutaneous pockets 140, 140′ and over which a detachable anti-microbial component characterized as a pad or boot that fits around at least a portion of an outer housing of the IMD is placed. The: subcutaneous site is advantageously protected from microbial growth and infections of the types described above by inclusion of the anti-microbial polymeric component that is exposed to body fluids in the pockets 140, 140′ that is compounded of an antibiotic zeolite that elutes silver ions in concentrations exhibiting anti-microbial activity over a substantial period of time of implantation. In these embodiments depicted in FIGS. 1-11, the anti-microbial component is physically attached to the IMD by fitting it over the IMD. It will be understood that the anti-microbial component can be molded to conform to the shape of any IMD adapted to be: implanted subcutaneously that is presently available or may become available in the future, e.g., gastric stimulators and drug pumps, insulin delivery drug pumps, and other body organ, muscle or nerve stimulators and drug delivery devices that are specifically identified herein. It will be further understood that an otherwise detachable anti-microbial component can be rendered substantially un-detachable by adhering the component to the IMD using, e.g., a medically acceptable adhesive.

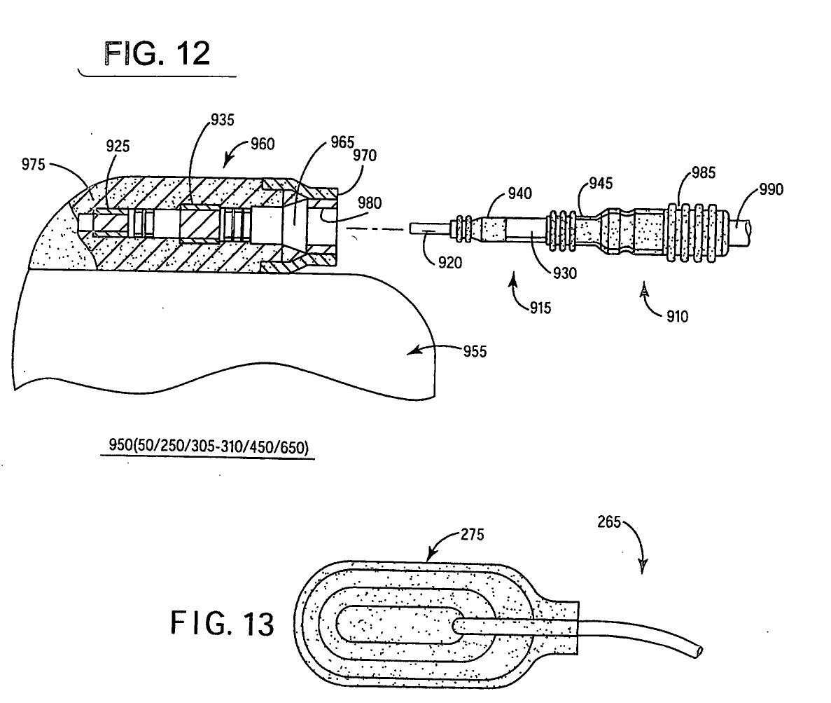

In an embodiment, the anti-microbial component comprises a permanently attached portion of any of the above-identified IMDs that are implanted into the prepared subcutaneous pocket 140. For example, a schematic partial view of an exemplary IPG/monitor 950 depicting the connector header 960 in partial cross section and an exemplary lead connector assembly 915 of an electrical medical lead 910 adapted to be fitted into a connector bore 965, is depicted in FIG. 12. Bipolar lead 910 is depicted having a connector assembly 915 of conventional bipolar design comprising a connector pin 920 and a connector ring 930 adapted to fit a pin receptacle contact 925 and a ring receptacle contact of schematically depicted connector header 960. Elastic polymeric sealing rings 940 and 945 are located adjacent to the connector pin 920 and connector ring 930. Distal portion 985 of the lead connector assembly 915 coupled to the elongated lead body 990 is disposed outside the connector bore 965 when the more proximal portion of the lead connector assembly 915 is fully inserted within the connector bore 965. Elastic bands 970 and 980 encircle the connector bore opening and a suture can be applied to tighten them against the elastic portion of the connector assembly between the sealing rings 945 and the distal portion 955. The particular configurations of the connector elements 925 and 935, the feedthroughs and wire connections, and any setscrews or other fasteners that are encased within the molded polymeric header body 975 for making secure electrical connections can take any of the known configurations and are not important to the practice of the present invention and are not depicted. The depicted IPG/monitor 950 is exemplary of any of the IPG/monitors and components thereof 50, 250, 305-310, 450, and 650, although the number of connector elements of the lead connector assembly and the connector header and their specific configurations may vary widely.

Selected ones or all of the polymeric components of the IPG connector header 975 and/or the lead connector assembly 915 are compounded with metal ion zeolite as indicated by the cross-hatching in FIG. 12 in accordance with a further embodiment of the invention. Usually, the lead connector assembly 915 is separately formed and attached to the lead body 990 in manufacture, so it is convenient to mold the polymeric lead connector assembly parts from silicone rubber or polyurethane compounded with the metal ion zeolite. The anti-microbial silver ions can thereby be eluted from the connector header body 975 and/or from the elastic band 970 and or from the lead connector portion 985 that is disposed outside the connector bore 965. The anti microbial silver ions can also be eluted from the sealing rings 940 and 945 if they become wet with body fluids over chronic implantation to inhibit any microbial activity within the connector bore/connector assembly interface.

FIG. 13 is a perspective view of a subcutaneously implantable electrode, e.g., electrode 275 wherein selected ones or all of the polymeric components of the electrode 275 are compounded with metal ion zeolite in accordance with a further embodiment of the invention. In particular, all or portions of the silicone rubber or polyurethane pad 220 can be molded with the metal ion zeolite as indicated by the cross-hatching in FIG. 13. Again, the silicone rubber or polyurethane pad 220 is separately formed and attached to the lead body of lead 265 in manufacture, so it is convenient to mold the polymeric pad as a single part or as multiple parts, depending on the design, from silicone rubber or polyurethane compounded with the metal ion zeolite.

Similarly, the polymeric header 860 of the implantable monitor 800, for example, the subcutaneously tunneled cable 315, for example, between subcutaneously implanted IMD components, and the polymeric component of the catheter connectors 560 and 760 with the implantable drug pumps 500 and 700, for example, can be molded from polymers compounded with metal ion zeolite.

All patents and publications referenced herein are hereby incorporated by reference in their entireties.

It will be understood that certain of the above-described structures, functions and operations of the above-described preferred embodiments are not necessary to practice the present invention and are included in the description simply for completeness of an exemplary embodiment or embodiments.

1. An anti-infective boot for an implantable medical device, the boot comprising:
a polymeric material having a shape configured to snuggly engage at a least a portion of the implantable medical device; and
a first anti-infective agent disposed in or on the polymeric material in an amount effective to prevent infection when the covering is disposed about the implantable medical device and implanted into a pocket of a patient.

2. The boot of claim 1, wherein the implantable medical device is selected from the group consisting of a cardiac pacemaker, a cardioverter/defibrillators, a neurostimulator, and a drug infusion pump, and wherein the polymeric material has a shape configured to snuggly engage at a least a portion of the cardiac pacemaker, the cardioverter/defibrillators, the neurostimulator, or the drug infusion pump.

3. The boot of claim 1, wherein the medical device is a pulse generator and the covering comprises a side opening to allow a portion of a housing of the pulse generator to serve as a return electrode.

4. The boot of claim 3, wherein the side opening has a size and shape that fits within a diameter defined by a zone of inhibition of the anti-infective agent in or on the polymeric material.

5. The boot of claim 4, wherein the diameter is determined by the zone of inhibition after thirty days of implantation.

6. The boot of claim 4, wherein the diameter is determined by the zone of inhibition after ninety days of implantation.

7. The boot of claim 1, wherein the polymeric material comprises silicone.

8. The boot of claim 1, wherein the anti-infective agent is an antibiotic.

9. The boot of claim 8, wherein the antibiotic is minocycline.

10. The boot of claim 8, wherein the antibiotic is rifampin.

11. The boot of claim 1, further comprising a second anti-infective agent disposed in or on the polymeric material in an amount effective to prevent infection when the covering is disposed about the implantable medical device and implanted into a pocket of a patient.

12. The boot of claim 11, wherein the first anti-infective agent is minocycline and the second anti-infective agent is rifampin.

13. The boot of claim 1, wherein the anti-infective agent is an antiseptic.

14. A system comprising:
an implantable medical device; and
the boot comprising:
(a) a polymeric material having a shape configured to snuggly engage at a least a portion of the implantable medical device; and
(b) an anti-infective agent disposed in or on the polymeric material in an amount effective to prevent infection when the covering is disposed about the implantable medical device and implanted into a pocket of a patient.

15. The system of claim 14, wherein the implantable medical device is selected from the group consisting of a cardiac pacemaker, a cardioverter/defibrillators, a neurostimulator, and a drug infusion pump.

16. The system of claim 15, wherein the anti-infective agent is minocycline.

17. The system of claim 15, wherein the anti-infective agent is rifampin.

18. The system of claim 14, further comprising a second anti-infective agent disposed in or on the polymeric material in an amount effective to prevent infection when the covering is disposed about the implantable medical device and implanted into a pocket of a patient.

19. The boot of claim 18, wherein the first anti-infective agent is minocycline and the second anti-infective agent is rifampin.

20. A method of preparing an anti-infective implantable medical device, comprising:
placing a boot about at least a portion of the implantable medical device to snuggly engage at least a portion of the implantable medical device,
wherein the boot comprises
(a) a polymeric material having a shape configured to snuggly engage at a least a portion of the implantable medical device and
(b) an anti-infective agent disposed in or on the polymeric material in an amount effective to prevent infection when the covering is disposed about the implantable medical device and implanted into a pocket of a patient.

21. The method of claim 20, wherein the method further comprises forming the boot to a shape configured to snuggly engage at a least a portion of the implantable medical device.

22. The method of claim 21, further comprising incorporating the anti-infective agent into the polymeric material of the boot.
